(19) United States
(12) Patent Application Publication
(10) Pub. No.: US 2019/0350808 A1
(43) Pub. Date: Nov. 21, 2019

(54) SAMPLE COLLECTION KIT FOR POSITIVE SAMPLE IDENTIFICATION

(71) Applicant: SIEMENS HEALTHCARE DIAGNOSTICS INC., TARRYTOWN, NY (US)

(72) Inventors: Eric Olson, North Salem, NY (US); Kathryn Wong, Stamford, CT (US); Christopher DiPasquale, Nyack, NY (US); Scott Salmon, Tenafly, NJ (US); Benjamin Pollack, Plainview, NY (US); Steven Bellofatto, Closter, NJ (US); Steven Madsen, New York, NY (US); Courtney Nicholls, Cranston, RI (US); Neil Berenholz, Nyack, NY (US)

(21) Appl. No.: 16/349,787

(22) PCT Filed: Nov. 14, 2017
(86) PCT No.: PCT/US17/61585

§ 371 Date: May 14, 2019

Related U.S. Application Data
(60) Provisional application No. 62/421,876, filed on Nov. 14, 2016.

(51) Int. Cl. A61J 1/14; A61B 5/15

(52) U.S. Cl. CPC A61J 1/14; A61B 5/150786; B01L 9/06; A61B 5/150343; B01L 2300/021; A61B 5/150351

(57) ABSTRACT

A sample collection kit is described for ensuring positive sample identification. The sample collection kit includes a sample container configured to hold a biological sample. The sample container has a lower end, an upper end, a sidewall that extends from the lower end to the upper end, and an identifier disposed along the sidewall. The identifier includes information encoded therein related to the sample container and the sample. The kit includes a sample container holder. The sample container holder has a bottom end, an open top end, a sidewall, and a window in the sidewall. The window is positioned in the sidewall of the container holder such that when the collection tube is inserted inside the sample container holder, the identifier of the sample container is aligned with window of the sample container holder.

### CROSS-REFERENCE TO RELATED APPLICATIONS

This application claims priority to and the benefit of U.S. Provisional Application Ser. No. 62/421,876, filed Nov. 14, 2016, the entire contents of which are incorporated by reference into the present application.

### TECHNICAL FIELD

The present disclosure relates to a sample collection kit and in particular to a sample collection kit used to facilitate positive sample identification.

### BACKGROUND

In clinical laboratory practice, it is important to maintain a positive link between the identity of the patient and the identity of a sample collected from that patient. Misidentification can occur if this link is erroneous or the link is lost, and this can result in significant medical errors and impact to patient health. It is critical that clinical laboratories establish procedures to minimize the likelihood of sample misidentification. The conventional method of minimizing the likelihood of sample misidentification is to apply an adhesive label to each sample tube at the time of collection. Labels may be printed or handwritten, and they may or may not include a machine-readable barcode. The identifiers on the label may include patient name, date of birth, hospital number, social security number, requisition number, accession number, or a unique random number. Standard practice in the United States includes recording two different identifiers on the label, in order to reduce misidentification errors related to patients who have an identifier in common. For example, two patients may have the same first and last name or the same date of birth. Two patients may also share the same identifier (requisition, accession) if the provider recycles the set of numbers they use. Typically, labels are applied to the specimens in the presence of the patient. Some providers verbally confirm the correctness of the label with the patient before and/or after collecting a sample, such as blood. In some cases, samples need to be relabeled after they arrive at the laboratory. This is necessary when the provider who collects the samples uses a different patient identification scheme from the laboratory performing the test, or when the provider cannot print barcodes which are compatible with the laboratory's instrumentation. While this standard practice is acceptable for purposes of laboratory accreditation, there are still numerous opportunities for mistakes that can result in sample misidentification. Further innovation in this field may reduce the likelihood of sample misidentification and reduce the amount of time and work needed to label samples.

### SUMMARY

An embodiment of the present disclosure is a sample collection kit. The sample collection kit includes a sample container configured to hold a sample of a biological fluid. The sample container has a lower end, an upper end, a sidewall that extends from the lower end to the upper end, and an identifier disposed along the sidewall. The identifier includes information encoded therein related to the sample container and the sample contained therein. The kit also includes a sample container holder configured to hold the sample container. The sample container holder has a bottom end, an open top end, a sidewall that extends from the bottom end to the open top end, and a window in the sidewall. The window is positioned in the sidewall of the container holder such that when the collection tube is inserted inside the sample container holder, the identifier of the sample container is aligned with the window of the sample container holder.

### BRIEF DESCRIPTION OF THE DRAWINGS

The foregoing summary, as well as the following detailed description, will be better understood when read in conjunction with the appended drawings. The drawings show illustrative embodiments of the disclosure. It should be understood, however, that the application is not limited to the precise arrangements and instrumentalities shown.

FIG. 1 is a schematic view of a sample collection kit according to an embodiment of the present disclosure;

FIG. 2 is an exploded view of a sample collection unit in the kit, including a sample container and the sample container holder 50 illustrated in FIG. 1;

FIG. 3 is a side view the sample collection unit shown in FIG. 1; and

FIG. 4 is a cross-section of the sample collection unit take along line 5-5 in FIG. 3.

### DETAILED DESCRIPTION OF ILLUSTRATIVE EMBODIMENTS

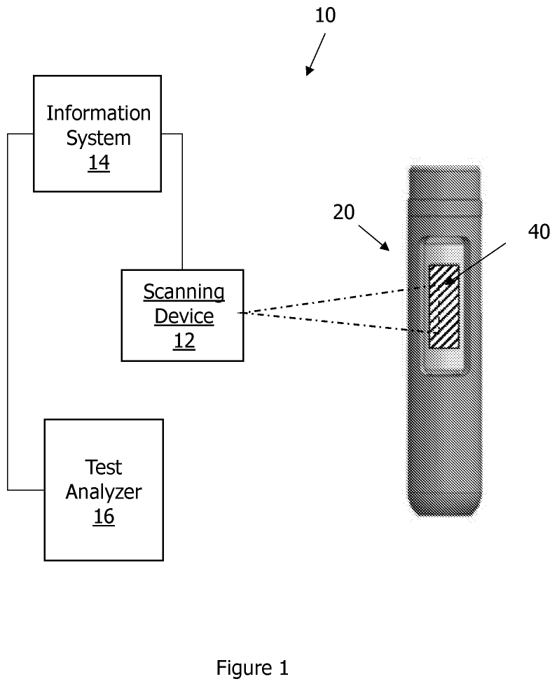

Embodiments of the present disclosure include a sample identification (ID) system 10. As shown in FIG. 1, the sample ID system 10 may include a sample collection unit 20, a scanning device 12, an information system 14, and a test analyzer 16. The sample collection unit 20 is configured to hold a sample, such as biological sample, and also to permit identification of the sample. The system may be implemented as a kit that includes the sample collection unit 20. The biological sample may be blood or some other biological fluid. A scanning device 12 is used to scan information contained in an identifier 40 on the sample collection unit 20, as will be further explained below. The information may be transmitted to the information system 14 and/or the test analyzer 16 whereby the analytical results performed by the test analyzer 16 are electronically associated with the sample contained in the sample collection unit 20.

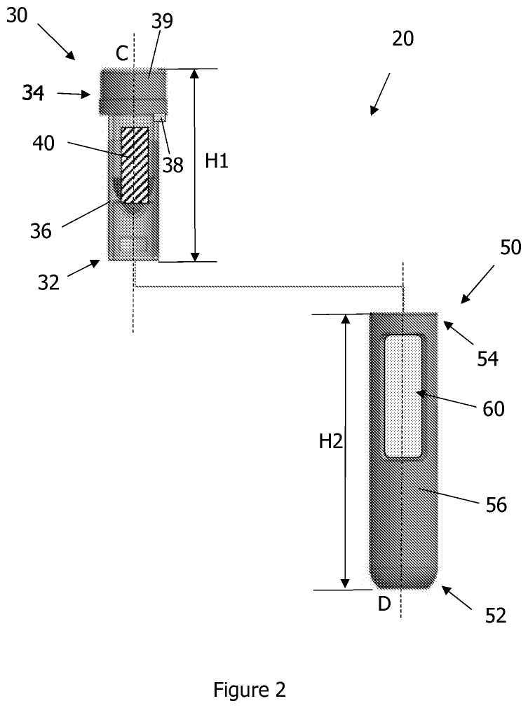

As shown in FIGS. 2-4, the sample collection unit 20 includes a sample container 30 and a sample container holder 50 that can hold and support the sample container 30. The sample container 30 is configured to hold a sample of the biological fluid. The sample container 30 has a lower end 32, an upper end 34, and a sidewall 36 that extends from the lower end 32 to the upper end 34. The sample container 30 extends along a central axis C, and has a height H1 that extends from the lower end 32 to the upper end 34 along the central axis C. The sample container 30 may also have one or more alignment features 38, such as ridge, dent, or groove, which is used to align the sample container 30 in the sample holder 50 as will be further described below.

The sample container 30 defines an internal volume that holds the biological fluid. In one example, the internal volume is sufficient to hold between 100 to 1000 μl of blood. In one example, the volume is between 300 and 600 μl. In another example, the volume is between 200 and 300 μl. For instance, the sample container 90 is sized to hold a micro-sample. For instance, the sample container 30 is sized to hold a micro-sample. However, in other embodiments, the sample container is sized to hold larger amounts of blood. As shown, the sample container 30 includes a cap 39 that can close off the upper end 34 of the sample container 30. The cap 39 is configured to couple to the open top end 54 of the sample container holder 50.

Continuing with FIGS. 2-5, the sample container 30 may also include an identifier 40 disposed along the sidewall 36. The identifier 40 has information encoded therein related to the sample container and the sample contained therein. The identifier 40 may be any machine readable image or design element positioned on the sample container 30. In one example, the identifier can be a code, an alphanumeric code, a bar code, a QR code, design image, or other element that is machine readable by the scanning device 12. The identifier 40 may be considered an electronic code that can be associated with collection of information concerning the sample that is stored in memory of the information system 14. The information can include a wide range of information associated with the sample being tested. For instance, the information may include, but is not limited to, sample type, sample tube type, sample volume, patient identifier, patient date of birth, hospital number, social security number, requisition number, accession number, a unique random number, a planned test module, laboratory conducting the text, and/or treating physician.

Referring to FIG. 2, the identifier 40 may be formed into the sample container 30 so that it is integral or monolithic with the sample container. For instance, the identifier 40 may be funned in the sidewall 36 during manufacture, such as during molding. In another example, the identifier 40 may be etched into or deposited onto the sidewall 36. Alternatively, the identifier 40 may be attached to the sample container 30 with an adhesive or some other means. In this example, the identifier 40 may be added during manufacture or at the sample collection site.

The sample container 30 may be formed from a wide range of materials suitable for holding biological samples. In one example, the sample container 30 may be formed of glass. In another example, the sample container 30 is a polymeric material. The sample container 30 may be at least partially transparent. Alternatively, the sample container may be opaque.

Continuing with FIG. 2, a sample container holder 50 is shown that is configured to hold the sample container 30. The sample container holder 50 has a bottom end 52, an open top end 54, and a sidewall 56 that extends from the bottom end 52 to the open top end 54. The sample container holder 50 also has a window 60 in the sidewall 36. The window 60 is positioned in the sidewall 56 so that when the sample container 30 is inserted inside the sample container holder 50, the identifier 40 of the sample container 30 is aligned with window 60 of the sample container holder 50. The window in the sample container holder may be a transparent material. In another embodiment, the window in the sample container may be cut-out in the sidewall 56, i.e. a void through which the identifier can be scanned. The sample container holder 50 extends along a central axis D, and has a height H2 that extends from the bottom end 52 to the open top end 54 along the central axis D. The sample container holder 50 may be formed from a wide range of suitable materials. In one example, the sample container 30 may be formed of polymer. The sample container holder may be formed from a transparent material or an opaque material. The advantage of transparent material is that it allows you to inspect the blood sample. The advantage of opaque material is that it allows you to make the sample container type more identifiable for example, because ae a gold holder may be used for a gold-top tube and a lavender holder for a lavender-top tube.

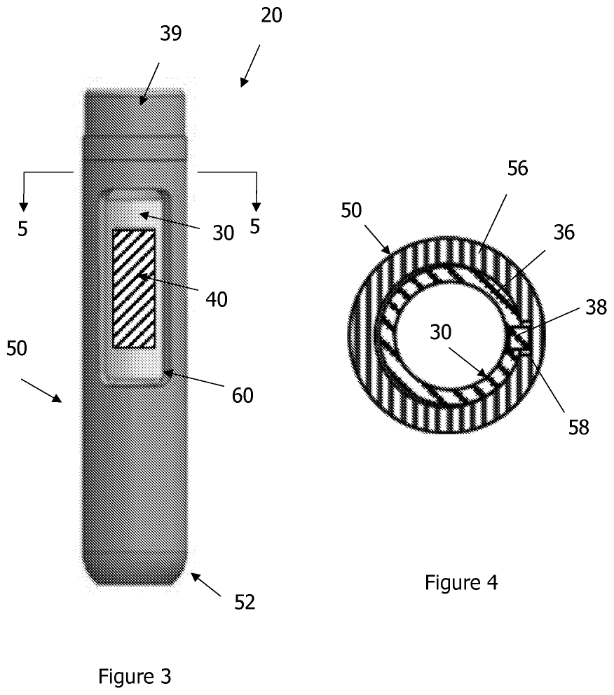

The sample container holder 50 may also have one or more alignment features, such as ridge, detent, or groove, which is used to align the sample container 30 in the sample holder 50. As illustrated in FIGS. 3 and 4, the sample collection unit 20 has alignment features that are used to align the identifier 40 of the sample container 30 with the window 60 in the sample container holder 50. As shown in FIGS. 2 and 4, the sample container 30 includes a first alignment feature 38 in the form of a ridge. Referring to FIG. 4, the sample container holder 50 includes a second alignment feature 58 in the form of a groove. The groove is sized to slidingly receive the ridge of the sample container 30. It should be appreciated that the first alignment feature 38 could be the groove and the second alignment feature could be the ridge. Furthermore, other types of alignment features may be used. The first and second alignment features 38, 58 are positioned so that the sample container 30 fits inside the sample container holder 50 at a particular height and orientation. As illustrated, the first and second alignment features 38, 58 mate with each other so that the identifier 40 is aligned with the window 60 both axially, along the height of the unit 20. In one example, the sample container has a first height H1 and the sample container holder 50 has a second height H2 such that the first height H1 is between 20% and 80% of the second height H2. The alignment features 38, 58 account for the difference in heights and help ensure that the identifier is properly aligned with the window. Furthermore, the first and second alignment features 38, 58 orient and fix the position of identifier 40 rotationally about the central axes C, D with respect to the sample container holder 50.

In an embodiment, the sample container 30 is configured to be fixed to the sample container holder 50. For instance, the collection unit may include a locking feature whereby the sample container couples irreversibly to the sample container holder.

Referring back to FIG. 1, the information system 14 may include one or more computing devices and software applications running thereon that control operation of the computer and process data obtained by the scanning device. The information system 14 may be any typical, networked system that operates multiple computers. The components of the information system 14 may be physically located together in similar physical site. Alternatively, the components of the information system may be distributed across multiple sites, such as multiple laboratories in different physical locations.

The scanning device 12 may be any electronic device configured to scan and/or read a machine readable code. For instance, the scanning device 12 may be a dedicated handset with a scanning module and a communications module. In such an example, the scanning module may be configured as a scanner that is a hand held device or one that is integrated with the test analyzer. The communications module transmits the scanned code to the information system 14 and/or the test analyzer 16. In another example, the scanning device 12 can be a handheld computing device, such as a smart phone, that is enabled to scan the identifier. In such an example, the handheld computing device may include a camera that captures an image of the identifier. Software in the handheld computing device associates the captured information with the stored information or a reference. The information system 14 can process the captured data as needed.

In operation, the sample is extracted from the subject with a lancet or needle into a capped sample container 30. Typically, the sample container will be initially capped and the sample will be put into the capped container via a cannula that goes through the cap The sample container 30 is placed in the sample container holder 50 so that the identifier is aligned with the window 60. The sample container holder 50 may be placed in the test analyzer 16, or a storage rack for later testing. The scanning device 12 may be used to scan the identifier 40 and the data processed and/or analyzed as needed. The sample container holder 50, 60 therefore permits the sample container 30 to be used with existing test equipment and sample handling device while also allowing the sample container 30 to be scanned without removing the sample container 30 from the sample container holder 50. There are other advantages. For instance, the use of container holder 50 as described herein allows the use of specimen containers without having to label the container holder 50. Ordinarily, labs either apply a new label to their container holder 50 when using small specimen containers, or they enter the sample ID into the analyzer manually. Furthermore, by using a unique identifier, errors related to two patients sharing a common identifier (e.g. same name, date of birth, provider ID) may be reduced. In some cases, for example, by applying the unique identifier at the time of manufacture, errors related to applying the wrong physical label to a specimen container are reduced or possibly eliminated. In addition, by positioning the identifier on the sample container at the time of manufacture, errors related to applying a physical label in such a way that the specimen container is unreadable or unusable is minimized. By using a container holder 50 which does not obscure the identifier on the specimen container, errors related to applying a new label to the container holder 50 are removed. Furthermore, when using a container holder 50 as described herein, which does not obscure the identifier on the specimen container, errors related to putting a specimen container into a container holder 50 whose identifier label does not match are reduced and/or eliminated. By using a sample container holder with different dimensions from the sample container, it is possible to have a sample container with optimal dimensions for sample collection, while also having a sample container with optimal dimensions for sample handling on automated systems.

While the present disclosure is described herein using a limited number of embodiments, these specific embodiments are not intended to limit the scope of the disclosure as otherwise described and claimed herein. Modification and variations from the described embodiments exist. It should be understood that the invention is not limited to the specific details set forth in the examples.

1. A sample collection unit, comprising:
a sample container configured to hold a biological sample, the sample container having a lower end, an upper end, a sidewall that extends from the lower end to the upper end, and an identifier disposed along the sidewall, the identifier including information encoded therein related to the sample container and the sample contained therein; and
a sample container holder configured to hold the sample container, the sample container holder having a bottom end, an open top end, a sidewall that extends from the bottom end to the open top end, and a window in the sidewall, the window positioned in the sidewall of the container holder such that when the collection tube is inserted inside the sample container holder, the identifier of the sample container is aligned with window of the sample container holder.

2. The sample collection unit of claim 1, wherein the sample container includes a first alignment feature and the sample container holder includes a second alignment feature configured to mate with the first alignment feature, wherein when the first and second alignment features mate, the identifier is aligned with the window.

3. The sample collection unit of claim 2, wherein one of the first alignment feature and the second alignment feature is a ridge and the other of the first alignment feature and the second alignment feature is a groove that is configured to receive the ridge.

4. The sample collection unit of claim 1, wherein the window is a transparent material.

5. The sample collection unit of claim 1, wherein the window is a cut-out of the sidewall of the sample container.

6. The sample collection unit of claim 1, wherein the sample container is configured to be fixed to the sample container holder.

7. The sample collection unit of claim 6, further comprising a locking feature whereby the sample container couples irreversibly to the sample container holder.

8. The sample collection unit of claim 1, wherein the biological sample is blood, and the sample container is sized to hold a microsample of blood.

9. The sample collection unit of claim 1, wherein a volume of the sample container is between 100 and 1000 μl.

10. The sample collection unit of claim 1, wherein the sample container has a central axis, and a first height that extends from the lower end to the upper end along the central axis, and the sample container holder has a second height that extends from the bottom end the open top end, wherein the first height is between 20% and 80% of the second height.

11. The sample collection of claim 1, wherein the sample container is at least partially transparent.

12. The sample collection unit of claim 1, wherein the sample container is opaque.

13. The sample collection unit of claim 1, wherein the sample container holder is at least partially transparent.

14. The sample collection unit of claim 1, wherein the sample container holder is opaque.

15. The sample collection unit of claim 1, further comprising a scanning device configured to scan the identifier on the sample container through the window of the sample container holder.

16. The sample collection unit of claim 1, further comprising a scanning device configured to scan the identifier on the sample container through the window of the sample container holder.
